(19) United States
(12) Patent Application Publication
(10) Pub. No.: US 2004/0110852 A1
(43) Pub. Date: Jun. 10, 2004

(54) Fischer-tropsch processes and catalysts using fluorided clay supports

(75) Inventors: Nithya Srinivasan, Ponca City, OK (US); Sergej A. Maslov, Ponca City, OK (US); Norman Herron, Newark, DE (US); Stephan Schwarz, Wilmington, DE (US); Munirpallam A. Subramanian, Kennett Square, PA (US)

Correspondence Address: DAVID W. WESTPHAL, CONOCOPHILLIPS COMPNAY, P.O. BOX 1267, PONCA CITY, OK, 74602-1267, US

(73) Assignee: ConocoPhillips Company, Houston, TX (US)

(21) Appl. No.: 10/309,454

(22) Filed: Dec. 4, 2002

(51) Int. Cl. C07C 27/06

(52) U.S. Cl. 518/715

(57) ABSTRACT

A process is disclosed for producing hydrocarbons. The process involves contacting a feed stream comprising hydrogen and carbon monoxide with a catalyst in a reaction zone maintained at conversion-promoting conditions effective to produce an effluent stream comprising hydrocarbons. In accordance with this invention, the catalyst used in the process includes at least a Fischer-Tropsch metal selected from Groups 8, 9, and 10 of the periodic table and combinations thereof. The catalyst also includes a fluorided clay support material. The fluorided clay is preferably a fluorided bentonite.

### RELATED APPLICATIONS

Not applicable.

### STATEMENT REGARDING FEDERALLY SPONSORED RESEARCH OR DEVELOPMENT

Not applicable.

### TECHNICAL FIELD OF THE INVENTION

The present invention relates to a process for the preparation of hydrocarbons from synthesis gas, i.e., a mixture of carbon monoxide and hydrogen, typically labeled the Fischer-Tropsch process. More particularly, this invention relates to a process including contacting synthesis gas with a catalyst containing a Fischer-Tropsch catalytic metal supported on a fluorided clay support, preferably a fluorided bentonite support.

### BACKGROUND OF THE INVENTION

Natural gas, found in deposits in the earth, is an abundant energy resource. For example, natural gas commonly serves as a fuel for power generation and as a fuel for domestic cooking. The process of obtaining natural gas from an earth formation typically includes drilling a well into the formation. Wells that provide natural gas are often remote from locations with a demand for the consumption of the natural gas.

Thus, natural gas is conventionally transported large distances from the wellhead to commercial destinations in pipelines. This transportation presents technological challenges due in part to the large volume occupied by a gas. Because the volume of a gas is so much greater than the volume of a liquid containing the same number of gas molecules, the process of transporting natural gas typically includes chilling and/or pressurizing the natural gas in order to liquefy it. However, this contributes to the final cost of the natural gas and is not economical.

Further, naturally occurring sources of crude oil used for liquid fuels such as gasoline and middle distillates have been decreasing and supplies are not expected to meet demand in the coming years. Middle distillates typically include heating oil, jet fuel, diesel fuel, and kerosene. Fuels that are liquid under standard atmospheric conditions have the advantage that in addition to their value, they can be transported more easily in a pipeline than natural gas, since they do not require energy, equipment, and expense required for liquefaction.

Thus, for all of the above-described reasons, there has been interest in developing technologies for converting natural gas to more readily transportable liquid fuels, i.e. to fuels that are liquid at standard temperatures and pressures. One method for converting natural gas to liquid fuels involves two sequential chemical transformations. In the first transformation, natural gas or methane, the major chemical component of natural gas, is reacted with oxygen to form syngas, which is a combination of carbon monoxide gas and hydrogen gas. In the second transformation, known as the Fischer-Tropsch process, carbon monoxide is reacted with hydrogen to form organic molecules containing carbon and hydrogen. Those organic molecules containing only carbon and hydrogen are known as hydrocarbons. In addition, other organic molecules containing oxygen in addition to carbon and hydrogen known as oxygenates may be formed during the Fischer-Tropsch process. Hydrocarbons having carbons linked in a straight chain are known as aliphatic hydrocarbons that may include paraffins and/or olefins. Paraffins are particularly desirable as the basis of synthetic diesel fuel.

The Fischer-Tropsch process is commonly facilitated by a catalyst. Catalysts desirably have the function of increasing the rate of a reaction without being consumed by the reaction. A feed containing carbon monoxide and hydrogen is typically contacted with the catalyst in a reactor. In a batch process, the reactor is closed to introduction of new feed and exit of products. In a continuous process, the reactor is open, with an inflow containing feed, termed a feed stream, passed into the reactor and an outflow containing product, termed a product stream, passed out of the reactor.

Typically the Fischer-Tropsch product stream contains hydrocarbons having a range of numbers of carbon atoms, and thus having a range of molecular weights. Thus, the Fischer-Tropsch products produced by conversion of natural gas commonly contain a range of hydrocarbons including gases, liquids and waxes. Depending on the molecular weight product distribution, different Fischer-Tropsch product mixtures are ideally suited to different uses. For example, Fischer-Tropsch product mixtures containing liquids may be processed to yield gasoline, as well as heavier middle distillates. Hydrocarbon waxes may be subjected to an additional processing step for conversion to liquid and/or gaseous hydrocarbons. Thus, in the production of a Fischer-Tropsch product stream for processing to a fuel it is desirable to obtain primarily hydrocarbons that are liquids and waxes, that is nongaseous hydrocarbons (e.g. C₅₊ hydrocarbons).

Typically, in the Fischer-Tropsch synthesis, the distribution of weights that is observed such as for C₅₊ hydrocarbons, can be described by likening the Fischer-Tropsch reaction to a polymerization reaction with a Shultz-Flory chain growth probability (α) that is independent of the number of carbon atoms in the lengthening molecule. α is typically interpreted as the ratio of the mole fraction of Cₙ₊₁ product to the mole fraction of Cₙ product. A value of α of at least 0.72 is desirable for producing high carbon-length hydrocarbons, such as those of diesel fractions.

The composition of a catalyst influences the relative amounts of hydrocarbons obtained from a Fischer-Tropsch catalytic process. Common catalysts for use in the Fischer-Tropsch process contain at least one metal from Groups 8, 9, or 10 of the Periodic Table (in the new IUPAC notation, which is used throughout the present specification).

Cobalt metal is particularly desirable in catalysts used in converting natural gas to heavy hydrocarbons suitable for the production of diesel fuel. Alternatively, iron, nickel, and ruthenium have been used in Fischer-Tropsch catalysts. Nickel catalysts favor termination and are useful for aiding the selective production of methane from syngas. Iron has the advantage of being readily available and relatively inexpensive but the disadvantage of a water-gas shift activity. Ruthenium has the advantage of high activity but is quite expensive. Consequently, although ruthenium is not the economically preferred catalyst for commercial Fischer-Tropsch production, it is often used in low concentrations as a reduction promoter with one of the other catalytic metals.

Catalysts often further employ a promoter in conjunction with the principal catalytic metal. A promoter typically improves a measure of the performance of a catalyst, such as productivity, lifetime, selectivity, reducibility, or regenerability. Further, in addition to the catalytic metal, a Fischer-Tropsch catalyst often includes a support material. The support is typically a porous carrier that provides mechanical support for the metal.

In a common method of loading catalytic metal to a support, the support is impregnated with a solution containing a dissolved catalytic metal-containing compound. After drying the support, the resulting catalyst precursor is calcined to decompose the catalytic metal-containing compound to an oxide compound of the catalytic metal. When the catalytic metal is cobalt, the catalyst precursor is then typically reduced in hydrogen to convert the oxide compound to reduced “metallic” metal.

Catalyst supports for catalysts used in Fischer-Tropsch synthesis of hydrocarbons have typically been refractory oxides (e.g., silica, alumina, titania, thoria, zirconia or mixtures thereof, such as silica-alumina). It has been asserted that the Fischer-Tropsch synthesis reaction is only weakly dependent on the chemical identity of the metal oxide support (see E. Iglesia et al. 1993, In: “Computer-Aided Design of Catalysts,” ed. E. R. Becker et al., p. 215, New York, Marcel Dekker, Inc.). Nevertheless, because it continues to be desirable to improve the activity of Fischer-Tropsch catalysts, other types of catalyst supports are being investigated.

In particular, aluminum silicate supports have been investigated. For example, bentonite is an aluminum silicate support that has been investigated in the Fischer-Tropsch reaction. Bentonite is a naturally occurring clay and thus is one of the catalyst supports that were investigated in the early years of Fischer-Tropsch research.

U.S. Pat. Nos. 6,075,062 and 6,121,190 disclose that patented systems based on cobalt include Co/MgO supported on bentonite (1958, M. W. Kellog).

U.S. Pat. No. 5,227,407 discloses that U.S. Pat. No. 2,539,847 relates to a Fischer-Tropsch hydrocarbon synthesis process employing a catalyst consisting of thoria promoted cobalt supported on bentonite.

British Patent 593,9840 discloses mineral acid activated bentonitic clay as a carrier for a Fischer-Tropsch catalyst. The catalyst further includes a Group VIII (Group 8, 9, or 10, in the new notation) metal and a difficultly reducible metal oxide promoter, such as an oxide selected from among thorium, magnesium, uranium, manganese, and aluminum. As is known in the art, in mineral acid activation, hydrogen ions are exchanged for positive metal ions, such as one or more ions of calcium or sodium, within the bentonite clay.

U.S. Pat. No. 4,831,060 discloses that mixed alcohols are produced from carbon monoxide and hydrogen gases using an easily prepared catalyst/co-catalyst system. The catalyst metals are molybdenum, tungsten or rhenium. The co-catalyst metals are cobalt, nickel or iron. The catalyst is promoted with a Fischer-Tropsch promoter like an alkali or alkaline earth series metal or a smaller amount of thorium and is further treated by sulfiding. The composition of the mixed alcohols fraction can be selected by selecting the extent of intimate contact among the catalytic components. U.S. Pat. No. 4,831,060 further discloses that the catalyst may be combined with binders such as bentonite clay, and/or pelleting lubricants such as Sterotex™ and formed into shapes for use as a finished catalyst.

Despite the above-described investigations of the use of bentonite-supported Fischer-Tropsch catalysts, the use of such a support has not obtained commercial favor. More recent investigations have tended to focus on catalysts supported on refractory metal oxides, such as silica, alumina, zirconia, and titania. These supports, typically synthetically made or obtained as processed derivatives of natural materials, have the advantage of more easily controlled physical properties. However, they have the disadvantage that the Fischer-Tropsch metal, particularly cobalt, tends to complex with the support under reaction conditions, becoming difficult to reduce, thus impeding regeneration of the catalyst. Thus, it has become the conventional practice to include reduction promoters, typically noble metals, such as rhenium, platinum, or ruthenium, to improve the reducibility of the catalytic metal. Noble metal promoters also typically improve the productivity of the catalyst. However, noble metal promoters have the disadvantage of contributing significantly to the cost of the catalyst.

Thus, notwithstanding the above teachings there remains a need for an improved Fischer-Tropsch catalyst system using an economical support, and a process using same, that is desirably active and/or selective for production of a hydrocarbon product including a diesel oil fraction, such as C₁₁-C₂₀ hydrocarbons.

### SUMMARY OF THE INVENTION

According to a preferred embodiment of the present invention, a process for producing hydrocarbons features converting a feed stream comprising carbon monoxide and hydrogen to a product stream comprising hydrocarbons in the presence of a catalyst that includes a fluorided clay support.

According to an alternative embodiment of the present invention, the process includes converting a feed stream comprising carbon monoxide and hydrogen to a product stream comprising hydrocarbons in the presence of a catalyst made by a method including providing a fluorided clay, loading at least one Fischer-Tropsch catalytic metal so as to form a catalyst precursor; and, activating the catalyst precursor so as to form the catalyst.

In some embodiments the clay includes a smectite. Alternatively, the clay may include a montmorillonite. Still alternatively, the clay may include a bentonite. The bentonite may be any suitable bentonite, including sodium bentonite, calcium bentonite, and the like.

In some embodiments, the catalyst includes a reduction promoter. Alternatively, in some other embodiments the catalyst excludes a reduction promoter and has at least essentially the same performance as a corresponding catalyst including a reduction promoter.

Thus, the present invention comprises a combination of features and advantages which enable it to overcome various problems of prior devices. The various characteristics described above, as well as other features, will be readily apparent to those skilled in the art upon reading the following detailed description of the preferred embodiments of the invention.

### DETAILED DESCRIPTION OF THE PREFERRED EMBODIMENTS

### Catalyst Support

According to a preferred embodiment of the present invention, an effective Fischer-Tropsch catalyst can include a fluorided clay support. In particular, supports that are contemplated for use with the present invention include smectite, montmorillonites, and bentonites. It will be understood that the smectites include the montmorillonites. Further, a major portion of bentonite is made up of montmorillonites.

A preferred support is fluorided bentonite. Fluorided bentonite is commercially available from catalyst suppliers, for example Engelhard.

Alternatively, any suitable process may be used for fluoriding a clay support, selected from processes for fluoriding a support. For example, a clay may be reacted with a vaporizable fluorine-containing compound. Suitable fluorine-containing compounds include HF, CCl₃F, CCl₂F₂, CHClF₂, CH₃CHF₂, CCl₂FCClF₂ and CHF₃.

By fluorided clay is meant a composition comprising oxygen, fluorine, aluminum, and silicon that has a clay structure. The fluorine content of the fluorided clay can vary over a wide range. Fluorided clays containing from 0.001% to about 10% by weight fluorine are preferred. The remainder of the fluorided clay component will include oxygen and aluminum and silicon. The fluorided clay may further include elements occurring naturally in clay or elements exchanged, by process known in the art for a naturally-occurring element.

Further, a fluorided clay may be based on a pillared clay. Pillared clays are known in the art and have the advantage of increased mechanical stability. Where the support includes a fluorided pillared clay, the fluorided pillared clay preferably is made by fluoriding a pillared clay. Further, the fluorine is preferably present as a surface component, the surface including pore structures.

The support may include fluorine in an amount sufficient to cause the support to be more acidic than neutral (pH=7) but less acidic than a zeolite cracking catalyst.

It will be appreciated that water is a byproduct of the Fischer-Tropsch reaction. Thus, the support material is preferably not water-swellable. For example, when the support material includes bentonite, the bentonite is preferably not a water-swellable bentonite.

### Catalyst Composition

The present catalyst preferably includes a catalytic metal. The catalytic metal is preferably a Fischer-Tropsch catalytic metal. In particular, the catalytic metal is preferably selected from the among the Group 8 metals, such as iron (Fe), ruthenium (Ru), and osmium (Os), Group 9 metals, such as cobalt (Co), rhodium (Rh), and irridium (Ir), Group 10 elements, such as nickel (Ni), palladium (Pd), and platinum (Pt), and the metals molybdenum (Mo), rhenium (Re), and tungsten (W). The catalytic metal is more preferably selected from the iron-group metals (i.e. cobalt, iron, and nickel), and combinations thereof. The catalytic metal still more preferably is selected from among cobalt and iron. The catalyst preferably contains a catalytically effective amount of the catalytic metal. The catalyst preferably contains a catalytically effective amount of the catalytic metal. The amount of catalytic metal present in the catalyst may vary widely.

When the catalytic metal is cobalt, the catalyst preferably has a nominal composition that includes cobalt in an amount totaling from about 1% to 50% by weight (as the metal) of total catalyst composition (catalytic metal, support, and any optional promoters), more preferably from about 5% to 40% by weight, still more preferably from about 10 to about 37 wt. % cobalt, sill yet more preferably from about 15 to about 35 wt. % cobalt. It will be understood that % indicates percent throughout the present specification.

It will be understood that, when the catalyst includes more than one supported metal, the catalytic metal, as termed herein, is the primary supported metal present in the catalyst. The primary supported metal is preferably determined by weight, that is the primary supported metal is preferably present in the greatest % by weight.

The catalytic metal contained by a catalyst according to a preferred embodiment of the present invention is preferably in a reduced, metallic state before use of the catalyst in the Fischer-Tropsch synthesis. However, it will be understood that the catalytic metal may be present in the form of a metal compound, such as a metal oxide, a metal hydroxide, and the like. The catalytic metal is preferably uniformly dispersed throughout the support. It is also understood that the catalytic metal can be also present at the surface of the support, in particular on the surface or within a surface region of the support, or that the catalytic metal can be non-homogeneously dispersed onto the support.

Optionally, the present catalyst may also include at least one promoter known to those skilled in the art. The promoter may vary according to the catalytic metal. A promoter may be an element that also, in an active form, has catalytic activity, in the absence of the catalytic metal. Such an element will be termed herein a promoter when it is present in the catalyst in a lesser wt. % than the catalytic metal.

A promoter preferably enhances the performance of the catalyst. Suitable measures of the performance that may be enhanced include selectivity, activity, stability, lifetime, reducibility, and resistance to potential poisoning by impurities such as oxygen and sulfur and nitrogen containing compounds. A promoter is preferably a Fischer-Tropsch promoter, that is an element or compound that enhances the performance of a Fischer-Trospch catalyst in a Fischer-Tropsch process.

Optionally, the catalyst essentially excludes noble metal promoters. Thus, the catalyst may essentially exclude rhenium, ruthenium, silver, and platinum. Further, such a catalyst may have at least essentially the same performance as a corresponding catalyst comprising at least one of rhenium, ruthenium, silver, and platinum.

It will be understood that as contemplated herein, an enhanced performance or a comparative performance of the present catalyst may be calculated according to any suitable method known to one of ordinary skill in the art. In particular, the enhanced or comparative performance may be given as a percent and computed as the ratio of the performance difference to the performance of a reference catalyst. The performance difference is between the performance of the present catalyst and the reference catalyst. The reference catalyst may be, e.g. a similar corresponding catalyst having the nominally same amounts, e.g. by weight percent, of all components except the promoter. It will further be understood that as contemplated herein, a performance may be measured in any suitable units. For example, when the performance is the productivity, the productivity may be measured in grams product per hour per liter reactor volume, grams product per hour per kilogram catalyst, and the like.

Suitable promoters vary with the catalytic metal and may be selected from Groups 1-15 of the Periodic Table of the Elements. A promoter may be in elemental form. Alternatively, a promoter may be present in an oxide compound. Further, a promoter may be present in an alloy containing the catalytic metal. Except as otherwise specified herein, a promoter is preferably present in an amount to provide a weight ratio of elemental promoter: elemental catalytic of from about 0.00005:1 to about 0.5:1, preferably, from about 0.0005:1 to about 0.01:1 (dry basis).

Further, when the catalytic metal is cobalt, suitable promoters include Group 1 elements such as potassium (K), lithium (Li), sodium (Na), and cesium (Cs), Group 2 elements such as calcium (Ca), magnesium (Mg), strontium (Sr), and barium (Ba), Group 3 elements such as scandium (Sc), yttrium (Y), and lanthanum (La), Group 4 elements such as (titanium) (Ti), zirconium (Zr), and hafnium (Hf), Group 5 elements such as vanadium (V), niobium (Nb), and tantalum (Ta), Group 6 elements such as molybdenum (Mo) and tungsten (W), Group 7 elements such as rhenium (Re) and manganese (Mn), Group 8 elements such as ruthenium (Ru) and osmium (Os), Group 9 elements such as rhodium (Rd) and iridium (Ir), Group 10 elements such as platinum (Pt) and palladium (Pd), Group 11 elements such as silver (Ag) and copper (Cu), Group 12 elements, such as zinc (Zn), cadmium (Cd), and mercury (Hg), Group 13 elements, such as gallium (Ga), indium (In), thallium (Tl), and boron (B), Group 14 elements such as tin (Sn) and lead (Pb), and Group 15 elements such as phosphorus (P), bismuth (Bi), and antimony (Sb). When the catalytic metal is cobalt, the promoter is preferably selected from among rhenium, ruthenium, platinum, palladium, boron, silver, and combinations thereof.

When the catalyst includes rhenium, the rhenium is preferably present in the catalyst in an amount between about 0.001 and about 5% by weight, more preferably between about 0.01 and about 2% by weight, most preferably between about 0.2 and about 1% by weight.

When the catalyst includes ruthenium, the ruthenium is preferably present in the catalyst in an amount between about 0.0001 and about 5% by weight, more preferably between about 0.001 and about 1% by weight, most preferably between about 0.01 and about 1% by weight.

When the catalyst includes platinum, the platinum is preferably present in the catalyst in an amount between about 0.00001 and about 5% by weight, more preferably between about 0.0001 and about 1% by weight, and most preferably between about 0.0005 and 1% by weight.

When the catalyst includes palladium, the palladium is preferably present in the catalyst in an amount between about 0.001 and about 5% by weight, more preferably between about 0.01 and about 2% by weight, most preferably between about 0.2 and about 1% by weight.

When the catalyst includes silver, the catalyst preferably has a nominal composition including from about 0.05 to about 10 wt % silver, more preferably from about 0.07 to about 7 wt % silver, still more preferably from about 0.1 to about 5 wt % silver.

When the catalyst includes boron, the catalyst preferably has a nominal composition including from about 0.025 to about 2 wt % boron, more preferably from about 0.05 to about 1.8 wt. % boron, still more preferably from about 0.075 to about 1.5 wt % boron.

As used herein, a nominal composition is preferably a composition specified with respect to an active catalyst. The active catalyst may be either fresh or regenerated. The nominal composition may be determined by experimental elemental analysis of an active catalyst. Alternatively, the nominal composition may be determined by numerical analysis from the known amounts of catalytic metal, promoter, and support used to make the catalyst. It will be understood that the nominal composition as determined by these two methods will typically agree within conventional accuracy.

Further, as used herein, it will be understood that each of the ranges, such as of ratio or weight %, herein is inclusive of its lower and upper values.

### Catalyst Preparation

The present catalysts may be prepared by any of the methods known to those skilled in the art. By way of illustration and not limitation, methods of preparing a supported catalyst include impregnating a catalyst material onto the support, extruding the support material together with catalyst material to prepare catalyst extrudates, and/or precipitating the catalyst material onto a support. Accordingly, the supported catalysts of the present invention may be used in the form of powders, particles, pellets, monoliths, honeycombs, packed beds, foams, and aerogels. The catalyst material may include any one or combination of a catalytic metal, a precursor compound of a catalytic metal, a promoter, and a precursor compound of a promoter.

The most preferred method of preparation may vary among those skilled in the art depending, for example, on the desired catalyst particle size. Those skilled in the art are able to select the most suitable method for a given set of requirements.

One method of preparing a catalyst by impregnating a catalyst material onto a support includes impregnating the support with a solution containing the catalyst material. Suitable solvents include water and organic solvents (e.g., toluene, methanol, ethanol, and the like). Those skilled in the art will be able to select the most suitable solvent for a given catalyst material. The catalyst material may be in the form of a salt of a catalytic metal or promoter element. Thus, one method of preparing supported metal catalyst is by incipient wetness impregnation of the support with a solution of a soluble metal salt. Incipient wetness impregnation preferably proceeds by solution of a cobalt compound in a minimal amount of solvent sufficient to fill the pores of the support. Alternatively, the catalyst material may be in the form of a zero valent compound of a catalytic metal or promoter element. Thus, another preferred method is to impregnate the support with a solution of zero valent metal such as cobalt carbonyl (e.g. Co₂(CO)₈, Co₄(CO)₁₂) or the like.

Another method of preparing a catalyst by impregnating a catalyst material onto a support includes impregnating the support with a molten salt of a catalytic metal or promoter. Thus, another method includes preparing the supported metal catalyst from a molten metal salt. One preferred method is to impregnate the support with a molten metal nitrate (e.g., Co(NO₃)₂·6H₂O). A promoter compound may be impregnated separately from any cobalt, in a separate step. Alternatively, a promoter compound may be impregnated simultaneously with, e.g. in the same solution as, at least a portion of the catalytic metal.

When a catalyst material is impregnated as a precursor of the material, e.g. a salt or zero valent compound, those skilled in the art will be able to selected the most suitable precursor.

By way of example and not limitation, suitable cobalt-containing precursor compounds include, for example, hydrated cobalt nitrate (e.g. cobalt nitrate hexadydrate), cobalt carbonyl, cobalt acetate, cobalt acetylacetonate, cobalt oxalate, and the like. Hydrated cobalt nitrate, cobalt carbonyl and cobalt acetate are exemplary of cobalt-containing precursor compounds soluble in water. Cobalt oxalate is soluble in acids or acidic solutions. Cobalt acetate and cobalt acetylacetonate are exemplary of cobalt-containing precursor compounds soluble in an organic solvent.

Suitable rhenium-containing precursor compounds soluble in water are preferred and include, for example, perrhenic acid, ammonium perrhenate, rhenium pentacarbonyl chloride, rhenium carbonyl, and the like.

Suitable ruthenium-containing precursor compounds soluble in water include for example ruthenium carbonyl, Ru(NH₃)₆·Cl₃, Ru(III)2,4-pentanedionoate, ruthenium nitrosyl nitrate, and the like. Water-soluble ruthenium-containing precursor compounds are preferred.

Suitable platinum-containing precursor compounds soluble in water include, for example, Pt(NH₃)₄(NO₃)₂ and the like. Alternatively, the platinum-containing precursor may be soluble in an organic solvent, such as platinum acetyl acetonate soluble in acetone.

Suitable boron-containing precursor compounds soluble in water include, for example, boric acid, and the like. Alternatively, the boron-containing precursor may be soluble in an organic solvent.

Suitable silver-containing precursor compounds soluble in water include, for example, silver nitrate (AgNO₃) and the like. Alternatively, the silver-containing precursor may be soluble in an organic solvent.

Suitable palladium-containing precursor compounds include palladium nitrate (Pd(NO₃)₂) and the like. Suitable palladium-containing precursor compounds soluble in an organic solvent include palladium dioxide (PdO₂), which is soluble in acetone, and the like.

The impregnated support is preferably treated to form a treated impregnated support. The treatment may include drying the impregnated support. Drying the impregnated support preferably occurs at a temperature between 80 and 150° C. Typically, drying proceeds for from 0.5 to 24 hours at a pressure of 1 to 75 atm, more preferably 1 to 10 atm, most preferably 1 atm.

Alternatively, or in combination, treating an impregnated support to form a treated impregnated support may include calcining the impregnated support. The calcination preferably achieves oxidation of any impregnated compound or salt of a supported material to an oxide compound of the supported material. When the catalytic metal includes cobalt, the calcination preferably proceeds at a temperature at least 200° C. Further, the calcination preferably proceeds at a temperature less than the temperature at which loss of support surface area is appreciable. It is believed that at temperatures above 900° C. loss of support surface area is appreciable. Typically, calcining proceeds for from 0.5 to 24 hours at a pressure of 1 to 75 atm, more preferably 1-10 atm, most preferably 1 atm.

The impregnation of catalytic metal and any optional promoter on a support may proceed by multistep impregnation, such as by two, three, or four impregnation steps. Each impregnation step may include impregnation of any one or combination of catalytic metal and promoter. Each impregnation step may be followed by any of the above-described treatments of the impregnated support. In particular, each step of impregnating the support to form an impregnated support may be followed by treating the impregnated support to form a treated impregnated support. Thus, a multistep impregnation may include multiple steps of drying and/or calcination.

Typically, at least a portion of the metal(s) of the catalytic metal component of the catalysts of the present invention is present in a reduced state (i.e., in the metallic state). Therefore, it is normally advantageous to activate the catalyst prior to use by a reduction treatment in the presence of a reducing gas at an elevated temperature. The reducing gas preferably includes hydrogen. Typically, the catalyst is treated with hydrogen at a temperature in the range of from about 75° C. to about 500° C., for about 0.5 to about 36 hours at a pressure of about 1 to about 75 atm. Pure hydrogen may be used in the reduction treatment, as may a mixture of hydrogen and an inert gas such as nitrogen, or a mixture of hydrogen and other gases as are known in the art, such as carbon monoxide and carbon dioxide. Reduction with pure hydrogen and reduction with a mixture of hydrogen and carbon monoxide are preferred. The amount of hydrogen may range from about 1% to about 100% by volume.

### Fischer-Tropsch Operation

A process for producing hydrocarbons preferably includes contacting a feed stream that includes carbon monoxide and hydrogen with the present catalyst. Alternatively or in combination, a process for producing hydrocarbons includes contacting a feed stream that includes carbon monoxide and hydrogen with a catalyst in reaction zone so as to produce hydrocarbons, where the catalyst is a catalyst made according to the present method.

The feed gas charged to the process for producing hydrocarbons includes hydrogen, or a hydrogen source, and carbon monoxide. H₂/CO mixtures suitable as a feedstock for conversion to hydrocarbons according to the process of this invention can be obtained from light hydrocarbons such as methane by means of steam reforming, partial oxidation, or other processes known in the art. If additional hydrogen is needed, it is preferably provided by free hydrogen, although some Fischer-Tropsch catalysts have sufficient water gas shift activity to convert some water and carbon monoxide to hydrogen and carbon dioxide, thus producing hydrogen for use in the Fischer-Tropsch process. It is preferred that the molar ratio of hydrogen to carbon monoxide in the feed be greater than 0.5:1 (e.g., from about 0.67 to 2.5). Preferably, when cobalt, nickel, and/or ruthenium catalysts are used, the feed gas stream contains hydrogen and carbon monoxide in a molar ratio of about 1.6:1 to 2.3:1. Preferably, when iron catalysts are used the feed gas stream contains hydrogen and carbon monoxide in a molar ratio between about 1.4:1 and 2.3:1. The feed gas may also contain carbon dioxide. The feed gas stream should contain a low concentration of compounds or elements that have a deleterious effect on the catalyst, such as poisons. For example, the feed gas may need to be pretreated to ensure that it contains low concentrations of sulfur or nitrogen compounds such as hydrogen sulfide, hydrogen cyanide, ammonia and carbonyl sulfides.

The feed gas is contacted with the catalyst in a reaction zone. Mechanical arrangements of conventional design may be employed as the reaction zone including, for example, plugged flow, continuous stirred tank, fixed bed, fluidized bed, slurry phase, slurry bubble column, reactive distillation column, or ebulliating bed reactors, among others, may be used. The size and physical form of the catalyst may vary, depending on the reactor in which it is to be used. Plug flow, fluidized bed, reactive distillation, ebulliating bed, and continuous stirred tank reactors have been delineated in “Chemical Reaction Engineering,” by Octave Levenspiel, and are known in the art, as are slurry bubble column. A suitable slurry bubble column is described, for example, in co-pending commonly assigned U.S. patent application Ser. No. 10/193,357, hereby incorporated herein by reference.

When the reaction zone includes a slurry bubble column, the column preferably includes a three-phase slurry. Further, a process for producing hydrocarbons by contacting a feed stream including carbon monoxide and hydrogen with a catalyst in a slurry bubble column, preferably includes dispersing the particles of the catalyst in a liquid phase comprising the hydrocarbons so as to form a two-phase slurry; and dispersing the hydrogen and carbon monoxide in the two-phase slurry so as the form the three-phase slurry. Further, the slurry bubble column preferably includes a vertical reactor and dispersal preferably includes injection and distribution in the bottom half of the reactor. Alternatively, dispersal may occur in any suitable alternative manner, such as by injection and distribution in the top half of the reactor.

The Fischer-Tropsch process is typically run in a continuous mode. In this mode, the gas hourly space velocity through the reaction zone typically may range from about 50 volumes/hour/volume expanded catalyst bed (v/hr/v) to about 10,000 v/hr/v, preferably from about 300 v/hr/v to about 2,000 v/hr/v. The gas hourly space velocity is defined at normal conditions where the pressure is 1 bar and the temperature is 0 degree centigrade. The reaction zone temperature is typically in the range from about 160° C. to about 300° C. Preferably, the reaction zone is operated at conversion promoting conditions at temperatures from about 190° C. to about 260° C. The reaction zone pressure is typically in the range of about 80 psia (552 kPa) to about 1000 psia (6895 kPa), more preferably from 80 psia (552 kPa) to about 600 psia (4137 kPa), and still more preferably, from about 140 psia (965 kPa) to about 500 psia (3447 kPa).

The products resulting from the process will have a great range of molecular weights. Typically, the carbon number range of the product hydrocarbons will start at methane and continue to about 50 to 100 carbons or more per molecule as measured by current analytical techniques. The process is particularly useful for making hydrocarbons having five or more carbon atoms especially when the above-referenced preferred space velocity, temperature and pressure ranges are employed.

The wide range of hydrocarbons produced in the reaction zone will typically afford liquid phase products at the reaction zone operating conditions. Therefore the effluent stream of the reaction zone will often be a mixed phase stream including liquid and vapor phase products. The effluent stream of the reaction zone may be cooled to condense additional amounts of hydrocarbons and passed into a vapor-liquid separation zone separating the liquid and vapor phase products. The vapor phase material may be passed into a second stage of cooling for recovery of additional hydrocarbons. The liquid phase material from the initial vapor-liquid separation zone together with any liquid from a subsequent separation zone may be fed into a fractionation column. Typically, a stripping column is employed first to remove light hydrocarbons such as propane and butane. The remaining hydrocarbons may be passed into a fractionation column where they are separated by boiling point range into products such as naphtha, kerosene and fuel oils. Hydrocarbons recovered from the reaction zone and having a boiling point above that of the desired products may be passed into conventional processing equipment such as a hydrocracking zone in order to reduce their molecular weight down to desired products such as middle distillates and gasoline. The gas phase recovered from the reactor zone effluent stream after hydrocarbon recovery may be partially recycled if it contains a sufficient quantity of hydrogen and/or carbon monoxide.

Without further elaboration, it is believed that one skilled in the art can, using the description herein, utilize the present invention to its fullest extent. The following exemplary embodiments are to be construed as illustrative, and not as constraining the scope of the present invention in any way whatsoever.

### EXAMPLES

### Catalyst Preparation

### Example 1

Bentonite (15 g, Engelhard 956A-5-1841-17) was dried in flowing air to 200° C. for 30 mins. The sample was flushed with nitrogen and then taken into glove box. The solid was mixed well with cobalt carbonyl (Co₂(CO)₈, 9 g). The mixture was placed in a clean quartz boat in a tube furnace and sealed and removed from the glove box. Dry nitrogen was allowed to flow through the tube using a water bubbler and the content of the tube were heated to 100° C. (drying). The temperature was held for 15 mins then ramped to 200° C. and held at that temperature for 30 mins (calcining). The resulting catalyst sample was cooled and taken into the glove box. A portion of the sample was sent for batch testing. The remainder of the sample was sent for fixed bed testing.

### Example 2

The procedure of Example 1 was used except a mixture of cobalt carbonyl (Co₂(CO)₈, 0.6 g) and ruthenium carbonyl (Ru₃(CO)₁₂, 2.1 mg) was used in place of cobalt carbonyl (Co₂(CO)₈, 9 g) and 1 g of bentonite was used in place of 15 g of bentonite.

### Example 3

The procedure of Example 1 was used except that cobalt carbonyl (Co₂(CO)₈, 0.6 g) and rhenium carbonyl (Re₂(CO)₁₀, 0.02 g) was used in place of cobalt carbonyl (Co₂(CO)₈, 9 g) and 1 g of bentonite was used in place of 15 g of bentonite.

### Example 4

The procedure of Example 1 was used except that cobalt carbonyl (Co₂(CO)₈, 0.6 g) and rhenium pentacarbonyl chloride (0.02 g) was used in place of cobalt carbonyl (Co₂(CO)₈, 9 g) and 1 g of bentonite was used in place of 15 g of bentonite.

### Example 5

The procedure of Example 2 was used except that that a different source of bentonite clay (1 g, Engelhard 956A-5-1841-15) was used.

### Example 6

The procedure of Example 3 was used except that a different source of bentonite clay (1 g, Engelhard 956A-5-1841-15) was used.

### Example 7

The procedure of Example 4 was used except that a different source of bentonite clay (1 g, Engelhard 956A-5-1841-15) was used.

### Example 8

Fluorinated F-20 bentonite clay (1g, Engelhard #965A-5-2112-37-1) was dried in flowing air at 200° C. for 1 hr. The dried fluorinated bentonite was cooled and transported into a glove box. In the glove box, the dried fluorinated bentonite was mixed with cobalt carbonyl (Co₂(CO)₈, 0.6 g) and then heated in a clean quartz boat in flowing nitrogen, using a bubbler on exit, to 100° C. (drying). The temperature was held for 15 mins then ramped to 200° C. and held at that temperature for 30 mins (calcining). The resulting catalyst sample was cooled and taken into a glove box and a portion sent for batch testing.

### Example 9

The procedure of Example 8 was used except that a mixture of cobalt carbonyl (Co₂(CO)₈, 0.6 g) and rhenium carbonyl (0.2 g) was used in place of the cobalt carbonyl alone.

### Example 10

The procedure of Example 8 was used except a mixture of 0.6 g cobalt carbonyl (Co₂(CO)₈, 0.6 g) and ruthenium carbonyl (2.1 mg) was used in place of cobalt carbonyl alone.

### Example 11

The procedure of Example 8 was used except that a different source of F-20 bentonite clay was used (1 g, Engelhard #965A-5-2112-39-1).

### Example 12

The procedure of Example 11 was used except that a mixture of cobalt carbonyl (Co₂(CO)₈, 0.6 g) and rhenium carbonyl (0.2 g) was used in place of the cobalt carbonyl alone.

### Example 13

The procedure of Example 11 was used except a mixture of cobalt carbonyl (Co₂(CO)₈, 0.6 g) and ruthenium carbonyl (2.1 mg) was used in place of cobalt carbonyl alone.

Batch Testing

Each of the catalyst samples was treated with hydrogen prior to use in the Fischer-Tropsch reaction. The catalyst sample was placed in a small quartz crucible in a chamber and purged with 500 sccm (8.3×10⁻⁶ m³/s) nitrogen at room temperature for 15 minutes. The sample was then heated under 100 sccm (1.7×10⁻⁶ m³/s) hydrogen at 1° C./minute to 100° C. and held at 100° C. for one hour. The catalysts were then heated at 1° C./minute to 400° C. and held at 400° C. for four hours under 100 sccm (1.7×10⁻⁶ m³/s) hydrogen. The samples were cooled in hydrogen and purged with nitrogen before use.

A 2 mL pressure vessel was heated at 225° C. under 1000 psig (6994 kPa) of H₂:CO (2:1) and maintained at that temperature and pressure for 1 hour. In a typical run, roughly 50 mg of the reduced catalyst and 1 mL of n-octane was added to the vessel. After one hour, the reactor vessel was cooled in ice, vented, and an internal standard of di-n-butylether was added. The reaction product was analyzed on an HP6890 gas chromatograph. Hydrocarbons in the range of C₁₁-C₅₀ were analyzed relative to the internal standard. The lower hydrocarbons were not analyzed since they are masked by the solvent and are also vented as the pressure is reduced.

The C₁₁₊ Productivity (g C₁₁₊/hour/kg catalyst) was calculated based on the integrated production of the C₁₁-C₄₀ hydrocarbons per kg of catalyst per hour. The logarithm of the weight fraction (Wn) per each carbon number (n) divided by the carbon number (n), ln(Wₙ/n) was plotted as the ordinate versus the carbon number (n) as the abscissa. From the slope of that plot, a value of α was obtained. As is known in the art, α is defined as the probability of hydrocarbon chain growth. The standard deviation for the C₁₁₊ Productivity is about±30 g/hr/kg-catalyst. Each of Groups A-D includes catalyst samples all prepared according to the same method apart from catalyst composition.

Each of Groups A and B contains results for comparable catalysts different in the amount and identity of any promoters and having a bentonite support. A comparison of the results for the examples in Group A demonstrates that Ru and Re each acts as a productivity promoter for a catalyst including cobalt supported on bentonite.

Each of Groups C and D contains results for comparable catalysts different in the amount and identity of any promoters and having a fluorided bentonite support. A comparison of the results for the examples in each of Groups C and D demonstrate that, surprisingly, neither Ru nor Re appreciably acts as a productivity promoter for a catalyst including cobalt supported on bentonite. The productivity for the unpromoted catalyst in each of Groups C and D is at least essentially the same as the productivity for the corresponding promoted catalysts Groups C and D, respectively.

Groups A and B differ in the source of bentonite. Group C has the same source as Group A. Group D has the same source as Group B. A comparison of the results for the examples in Group D with those in Group C coupled with a comparison of the results for the examples in Group B with those in Group A demonstrates that the performance of the catalysts is essentially independent of the source of bentonite.

TABLE 1

| | | | |
|---|---|---|---|
| Example | Catalyst Nominal Composition | C₁₁₊ Productivity | □ |

Group A

| | | | |
|---|---|---|---|
| 1 | 16% Co/Bentonite | 220 | 0.90 |
| 2 | 16% Co/0.1% Ru/Bentonite | 280 | 0.89 |
| 3 | 16% Co/1% Re/Bentonite | 260 | 0.89 |
| 4 | 16% Co/1% Re/Bentonite | 380 | 0.89 |

Group B

| | | | |
|---|---|---|---|
| 5 | 16% Co/0.1% Ru/Bentonite | 270 | 0.89 |
| 6 | 16% Co/1% Re/Bentonite | 340 | 0.90 |
| 7 | 16% Co/1% Re/Bentonite | 340 | 0.89 |

Group C

| | | | |
|---|---|---|---|
| 8 | 16% Co/Bentonite(F) | 270 | 0.89 |
| 9 | 16% Co/1% Re/Bentonite(F) | 180 | 0.88 |
| 10 | 16% Co/0.1% Ru/Bentonite(F) | 280 | 0.89 |

Group D

| | | | |
|---|---|---|---|
| 11 | 16% Co/Bentonite(F) | 280 | 0.90 |
| 12 | 16% Co/1% Re/Bentonite(F) | 200 | 0.88 |
| 13 | 16% Co/0.1% Ru/Bentonite(F) | 260 | 0.88 |

Should the disclosure of any of the patents and publications that are incorporated herein conflict with the present specification to the extent that it might render a term unclear, the present specification shall take precedence.

While a preferred embodiment of the present invention has been shown and described, it will be understood that variations can be made to the preferred embodiment without departing from the scope of, and which are equivalent to, the present invention. For example, the structure and composition of the catalyst can be modified and the process steps can be varied.

The foregoing detailed description and examples have been given for clarity of understanding only. No unnecessary limitations are to be understood therefrom. The invention is not limited to the exact details shown and described, for variations obvious to one skilled in the art will be included within the invention. For example, the structure and composition of the catalyst can be modified and the order of process steps may be varied. Further, while the examples have been described with respect to a batch process, the process for producing hydrocarbons may be carried out in continuous mode. Accordingly, the scope of protection is not limited to the embodiments described herein, but is only limited by the claims that follow, the scope of which shall include all equivalents of the subject matter of the claims.

We claim:

1. A process for producing hydrocarbons, comprising contacting a feed stream comprising hydrogen and carbon monoxide with a catalyst in a reaction zone; said catalyst comprising at least one Fischer-Tropsch catalytic metal supported on a carrier comprising a fluorided clay.

2. The process according to claim 1 wherein said catalytic metal comprises cobalt.

3. The process according to claim 2 wherein said catalyst essentially excludes rhenium, ruthenium, silver, and platinum and has at least essentially the same performance as a corresponding catalyst comprising at least one of rhenium, ruthenium, silver, and platinum.

4. The process according to claim 2 wherein said catalyst further comprises a noble metal promoter.

5. The process according to claim 4 wherein said noble metal promoter is selected from the group consisting of rhenium, ruthenium, silver, and platinum.

6. The process according to claim 1 wherein said clay comprises a smectite.

7. The process according to claim 1 wherein said clay comprises a montmorillonite.

8. The process according to claim 1 wherein said clay comprises bentonite.

9. The process according to claim 8 wherein said bentonite comprises calcium bentonite.

10. The process according to claim 8 wherein said bentonite comprises sodium bentonite.

11. The process according to claim 8 wherein said bentonite comprises an acid-activated bentonite.

12. The process according to claim 1 wherein said carrier comprises fluorine in an amount sufficient to cause the support to be more acidic than neutral (pH=7) but less acidic than a zeolite cracking catalyst.

13. A process for producing hydrocarbons, comprising contacting a feed stream comprising hydrogen and carbon monoxide with a catalyst in a reaction zone; said catalyst comprising cobalt, a support selected comprising fluorided bentonite, and excluding ruthenium, rhenium, silver, and platinum, and wherein said catalyst has at least essentially the same performance as a corresponding catalyst comprising at least one of rhenium, ruthenium, and platinum.

14. The process according to claim 13 wherein said bentonite comprises calcium bentonite.

15. The process according to claim 13 wherein said bentonite comprises sodium bentonite.

16. The process according to claim 13 wherein said bentonite comprises an acid-activated bentonite.

17. A catalyst comprising at least one Fischer-Tropsch catalytic metal supported on a carrier comprising a fluorided clay.

18. The catalyst according to claim 17 wherein said clay comprises a smectite.

19. The catalyst according to claim 17 wherein said clay comprises a montmorillonite.

20. The catalyst according to claim 17 wherein said clay comprises bentonite.

21. The catalyst according to claim 20 wherein said bentonite comprises calcium bentonite.

22. The catalyst according to claim 20 wherein said bentonite comprises sodium bentonite.

23. The process according to claim 20 wherein said bentonite comprises an acid-activated bentonite.

24. The catalyst according to claim 17 wherein said catalytic metal comprises cobalt.

25. The catalyst according to claim 24 wherein said catalyst further comprises a promoter selected from the group consisting of rhenium, ruthenium, silver, and platinum.

26. The catalyst according to claim 24 wherein said catalyst excludes rhenium, ruthenium, silver, and platinum.

27. The catalyst according to claim 17 wherein said carrier comprises fluorine in an amount sufficient to cause the support to be more acidic than neutral (pH=7) but less acidic than a zeolite cracking catalyst.

28. The catalyst according to claim 17 wherein said catalyst is made by a method comprising:
(a) providing the fluorided clay;
(b) loading the Fischer-Tropsch catalytic metal so as to form a catalyst precursor; and
(c) activating said catalyst precursor so as to form said catalyst.

29. A method for making a catalyst, the method comprising:
(a) providing a fluorided clay;
(b) loading at least one Fischer-Tropsch catalytic metal so as to form a catalyst precursor; and
(c) activating said catalyst precursor so as to form said catalyst.

30. The method according to claim 29 wherein said clay comprises a smectite.

31. The method according to claim 29 wherein said clay comprises a montmorillonite.

32. The method according to claim 29 wherein said clay comprises bentonite.

33. The method according to claim 32 wherein said bentonite comprises calcium bentonite.

34. The method according to claim 32 wherein said bentonite comprises sodium bentonite.

35. The method according to claim 32 wherein said bentonite comprises an acid-activated bentonite.

36. The method according to claim 29 wherein said carrier comprises fluorine in an amount sufficient to cause the support to be more acidic than neutral (pH=7) but less acidic than a zeolite cracking catalyst.

37. The method according to claim 29 wherein step (b) comprises:
(b1) loading at least a first portion of said catalytic metal to said fluorided clay to as to form an intermediate catalyst precursor;
(b2) calcining said catalyst precursor; and
(b3) loading at least a second portion of said catalytic metal to said fluorided clay so as to form said catalyst precursor activated in step (c).
